(19) United States
(12) Patent Application Publication
(10) Pub. No.: US 2020/0315127 A1
(43) Pub. Date: Oct. 8, 2020

(54) EEG SWIVEL-BALANCE CAGE SYSTEM AND METHODS OF USE

(71) Applicant: American University of Beirut, Beirut (LB)

(72) Inventors: Makram Obeid, Beirut (LB); Rabih Hashash, Beirut (LB); Laura Dosh, Beirut (LB); Yasser Medlej, Beirut (LB)

(21) Appl. No.: 16/378,009

(22) Filed: Apr. 8, 2019

(51) Int. Cl. A01K 1/03; A61B 5/00

(52) U.S. Cl. CPC A01K 1/031; A01K 2267/0356; A01K 2227/105; A61B 5/0006

(57) ABSTRACT

Herein a Swivel-Balance Cage System is described that markedly improves the safety and longevity of continuous long-term EEG monitoring in rodents. The described customized swivel-balance cage system allows tension-free rodent mobility.

### BACKGROUND

The invention generally relates to a system and related method for conducting research on animals.

Electroencephalography (EEG) is a widely used technique and the main tool employed in the characterization of electrographic patterns and seizures for the diagnosis and treatment of acquired and genetic human epileptic disorders (1-3). In the experimental arena, the EEG is also an essential tool used in preclinical research to study electrographic changes and seizures in animal models that echo human diseases including rodent models of traumatic brain injury (TBI) and epilepsy (4-6). The ability to record continuous EEGs with or without video in small animals, commonly in rodents, over weeks to months is essential to model human diseases, and to study the natural history of acquired or genetic epilepsies. This is particularly true for conditions such as TBI where the chronobiology of seizure emergence remains poorly understood (7,8). Investigating desperately needed novel anti-seizure and neuroprotective treatment strategies against acquired epilepsy is one area of translational research that is the focus of many laboratories including ours (6,9,10). This specific area of research heavily relies on the ability to efficiently record continuous long-term EEG on multiple animals simultaneously in order to assess the effect of novel interventions and drugs against the emergence of chronic spontaneous recurrent seizures; a process referred to as epileptogenesis (11). More recently, long-term EEG data is also being increasingly subjected to fine computational analyses, mostly in an attempt to detect electrographic biomarkers that may predict seizure emergence (12).

Unfortunately, performing safe and prolonged EEG in rodents is complicated by a slew of challenges, the most notorious of which is disconnections of the EEG cable from the rodent's head. Disconnections are due to the high mobility of the tethered rodents with resulting tension on the EEG cables, as well as to their natural tendency to chew on these cables (5). This long-standing challenge to investigators not only compromise costly experiments, but studies may be terminated early and rodents may be injured, especially when they experience vigorous seizures. Some of the most widely used pre-clinical rodent models for studying chronic epilepsy after brain injury include hypoxia, chemoconvulsant administration, and electrical kindling (13-16). These epileptogenic brain insults often lead to vigorous and prolonged seizures known as status epilepticus, which is followed by the emergence of spontaneous seizures after weeks to months (chronic epileptic phase). Vigorous convulsions associated with the initial status epilepticus or the subsequent seizures in the chronic phase put the animals at a high risk of injury secondary to the pulling tension that they exert on EEG cables. While high quality prolonged video-EEG recordings are even more needed when animals experience seizures, the risk of motion artifact and premature experimental termination is also the greatest during that time. Early termination of experiments results in loss of data, animal injuries, and inflicts losses on investigators' time, energy, and budget. In addition to durability-related issues, another common technical challenge is the often poor signal to noise ratio. Excessive artifact is usually due to environmental electrical noise, animal movements, and difficulties in electrically shielding the animals. This is especially true when simultaneously monitoring multiple rodents with the medical EEG headboxes used in humans and thus designed to process, ground and reference EEG signal from one individual subj ect.

Despite the advances in wireless EEG technologies, “wired” EEG remains the most widely employed technique in rodents due to the usually limited number of implantable electrodes in wireless transmission technologies, as well as to the excessive cost of wireless transmitters (17). This cost issue is compounded by the fact that cohorts with large number of animals are needed in translational research, and consequently, a large number of expensive wireless transmitters.

The present invention attempts to solve these problems, as well as others.

### SUMMARY OF THE INVENTION

Provided herein are systems, methods and compositions for a swivel-balance cage system and methods of use. The swivel-balance cage system markedly ameliorates the success rate, longevity and quality of long-term “wired” EEG. The swivel-balance cage system comprises a vertically pivoting swivel that accommodates an animals movements in both the horizontal and vertical planes, and prevent premature disconnections and to confer resistance against wear and tear from cable torsion and tension.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIGS. 1A-1B are side views of the Swivel-Balance Cage System, where FIG. 1A is the vertically pivoting swivel mechanism without weights; FIG. 1B is the vertically pivoting swivel mechanism deployed with weights; FIG. 1C is a schematic showing when the swivel reaches its highest position outside the cage, when the swivel in, and when the swivel is out, and the associated calculations for each.

FIG. 2A is a perspective view of the customized plexiglas cage and its various parts, including the gridded floor and the removable ceiling. FIG. 2B is a side view showing a schematic representation of the sliding drawer.

FIGS. 3A-3B are top views of the surgical electrodes implantation, the headset assembly, and an illustration of a set of epidural screw electrode placement in a P35-40 rat. FIG. 3A shows five screw electrodes including two frontal (F3: left frontal, F4: right frontal), two parietal (P3: left parietal, P4: right parietal) and one anterior reference electrode (Ref). The coordinates are based on the Sherwood and Timiras stereotaxic atlas of the developing brain. Tightly anchoring the screws is key to prevent premature headset and EEG cable detachment from the skull. A sixth electrode consists of a free wire placed under the neck's skin and serves as a ground electrode. All five screw electrodes and the ground one are soldered to a golden plated socket (black arrow) via an insulated copper wire. The wires are lightweight and moldable in various shapes which facilitates handling during surgery and giving the headset its final shape. FIG. 3B is the 6 channel plastic pedestal (white arrow) that accommodates the 6 electrode sockets. After placing the sockets in their respective ports, the pedestal is pushed down while tucking in the copper wires in order to form a compact headset. FIG. 3C shows the dental cement poured from the sides using a syringe, while still in a semi-liquid form in order to allow infiltration of all the gaps between the wires and pedestals. This gives the protective headset its final form that keeps all the elements together, and protects the skull, without requiring sutures. FIG. 3D shows the EEG cables (white arrow) ending with 6 pins that fit into the 6 channel pedestal. The 6 pin EEG cable, a pedestal, and a screw electrode connected to a socket, are shown next to each other for illustrative reasons.

FIG. 4 is a side view of the EEG rack system. Each rack houses at least 4 cages, two on each shelf. Every 2 racks (8 cages) are monitored by one EEG recorder.

FIG. 5 is a side view of the swivel.

FIG. 6A is perspective view of the entire recording system. FIG. 6B is a schematic illustration of the full two rack system showing the EEG cable coming through the swivel is connected to an output relay box placed in the middle of the pivoting rectangular plate, centered over the hinge.

FIG. 7 is a screenshot of a continuous EEG recording performed simultaneously on 8 rats using one EEG recorder.

FIG. 8 is an illustrative image of a tracing in an average referential montage (panel A) and in a longitudinal bipolar montage (panel B). Left hemispheric spikes are seen with a maximum in the left parietal region (black rectangle). F3: left frontal, F4: right frontal, P3: left parietal, P4: right parietal, Avg: average of the 4 electrodes.

FIG. 9A are illustrative successive screenshots (A to C) of a right frontal seizure in an average referential montage (FIG. 9A) and in a longitudinal bipolar montage (FIG. 9B). A typical seizure with “a rhythmicity that evolves in space and time” is shown with clarity in this rat. The seizure starts in the right frontal area (black arrow) as shown in screenshot A in both FIGS. 9A-9B, then progresses to involve the right hemisphere with slower rhythmic activity (screenshot B in both FIGS. 9A-9B), and finally clearly involves the left hemisphere with bilateral rhythmic spikes (screenshot C in both FIGS. 9A-9B). The whole seizure is shown in FIG. 9C (white arrows indicate onset and offset). F3: left frontal, F4: right frontal, P3: left parietal, P4: right parietal, Avg: average of the 4 electrodes.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values are merely intended to serve as a shorthand method of referring individually to each separate value falling within the range, unless otherwise indicated, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” does not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

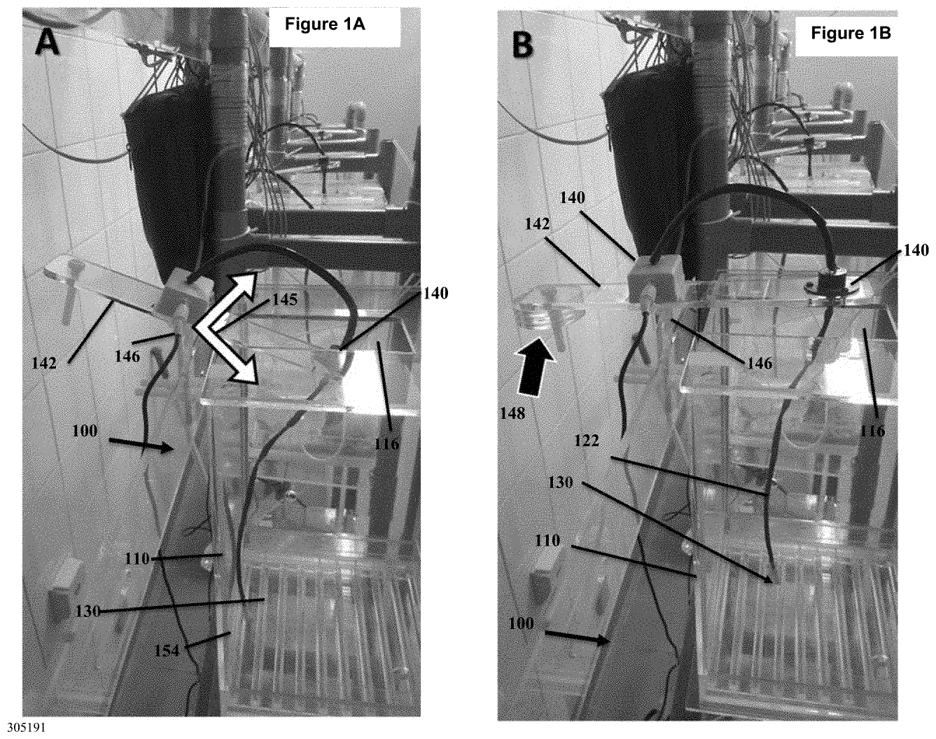

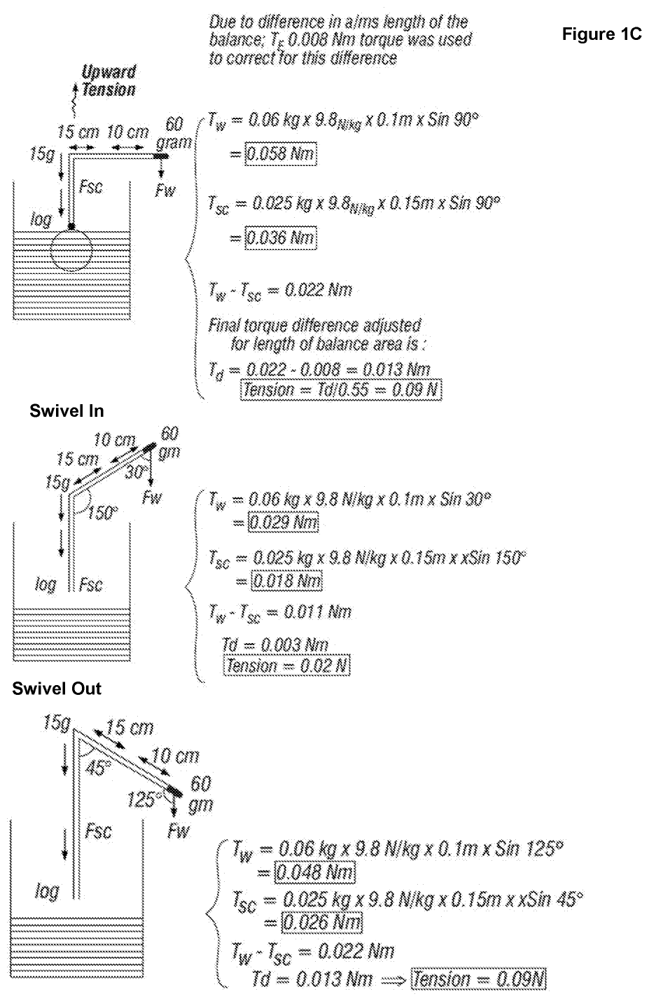

Translational epilepsy research heavily relies on electroencephalography (EEG) recording to investigate seizure mechanisms and chronobiology, and to test novel anti-seizure treatments in animal models. However, obtaining prolonged wired EEG can be challenging due to the small size of the rodents, their tendency to chew on EEG cables, their regular movements, and the often vigorous and potentially frequent seizures causing premature EEG cable detachments. This often leads to animal scalp injury and infection, as well as costly inefficient failures or premature terminations of experiments. To overcome these usual challenges faced with wired EEG techniques, an EEG Swivel-Balance Cage System is for long-term rodent EEG monitoring, as shown in FIG. 1.

The Swivel-Balance Cage System 100 prevents tension and torsion on the EEG cable as the rat freely moves in both the horizontal and vertical planes. A swivel mechanism is fixed to a mobile rectangular plate that can move up and down through the cage's ceiling as it pivots along a hinge (Panel A. double arrows). The cage is placed on the shelves so that the swivel is centered over it. The swivel is counterbalanced by circular weights (FIG. 1B, 148) placed at the other end of the plate to provide a counterbalance system. The circular weights are loosely screwed via non-concentric holes allowing easy balancing by just turning the weights around which alters the torque they exert on the plate to the desired effect. Prior to attaching the animal to the cable, the weights are calibrated so that the counterbalance arm holding the weights is slightly heavier (Panel B). This will provide a minimal continuous upward pull on the headset when the animal is attached, and prevent the swivel from remaining inside the cage when no downward tension is exerted by the animal, as occurs when the rat rises. The simple yet key modification allows long-term continuous EEG monitoring without premature disconnections of the EEG cable from the rat's head or other animal's head.

Besides electroencephalography (EEG), signal, the system of the present invention, if expanded, can accommodate the continuous recording of many other types of electrical biosignals that are important in biomedical research including, but not limited to: magnetoencephalogram (MEG), galvanic skin response (GSR), electrocardiogram (ECG), electromyogram (EMG), electrooculogram (EOG), Heart Rate Variability (HRV), etc. To record electrical biosignals, an appropriate telemetry transmitter is placed into the cage. Besides electrical biosignals, recording other types of biological signals can be performed by this system, if a telemetry transmitter for recording such signal is available. Moreover, a telemetry transmitter may not be needed if a signal is recorded by a device that fits into the container. For example, recording of body or brain temperature can be done using a miniature temperature logger with external sensor connected with the animal. This type of logger records temperature data into its internal memory, so that the data can be subsequently transferred to a computer. Other examples of biological signals that can be recorded by this system include continuous monitoring of tissue oxygen, breath rate, pulse distention, arterial pressure, left ventricular pressure, renal sympathetic nerve activity. Currently, there are many commercially available systems that allow such measurements using telemetry transmitters that are sufficiently small to fit in the container of system of the present invention. Data Sciences International (DSI) offers telemetry transmitters for recording EEG, EMG and temperature in both rats and mice. Both the mouse and rat transmitter are sufficiently small to be used in the system of the present invention. Millar Telemetry System offers telemetry transmitters for recording EEG, EMG, tissue oxygen, arterial pressure, left ventricular pressure, and renal sympathetic nerve activity. They use a telemetry transmitter that weighs 12-13 g and has volume of 7 ml. Signal artifacts lead to more laborious scoring and analysis and limit the number of epochs sampled providing less reliable results and potentially missing valuable data. The high-quality EEG signals recorded with the new system allows for enhanced data sampling and reduced EEG power variability. At the same time, the herein described system is much less expensive than the advanced wireless technologies, and consumes less power.

The system of the present invention is particularly useful for studies with small laboratory animals such as rats, mice or hamsters. However, there are some studies in which larger animals might be preferred. In these cases, the system can be easily modified to allow recording in a larger animal. The modification will require a stronger first cable and a stronger arm that would withstand the force of a larger animal. Thus, the system of the present invention provides for the first time a relatively inexpensive and reliable means of interface for interacting with a laboratory animal of various sizes using laser light, fluids or electrical signals.

Therefore, there is provided a system that is easy to use, fits standardized laboratory cages, allows for more consistent generation of data due to reduced data artifacts and less stress induced on the animals, provides the ability for more normal activity patterns to be conducted by the animals, allows for better utilization of automated scoring due to improved biopotential signal recordings, and improves the reliability and accuracy of drug and optogenetics stimuli generation.

The Swivel-Balance Cage System prevents tension and torsion on the EEG cable as the rat freely moves in both the horizontal and vertical planes. Tension may be described as the pulling force transmitted axially by the means of a cable 116; tension might also be described as the action-reaction pair of forces acting at each end of the cable 122. The gentle upward tension that the balance-system exerts on the EEG cable 122 inside the cage is estimated to be 0.09 N when the balance is in the horizontal plate position and down to 0.02 N when the plate is all the way down inside the cage. The commutator initiates rotation at a torque value of 0.06 Nm. This low swivel torque value prevents any EEG cable torsion (the twisting of the cable due to an applied torque). A swivel mechanism 140 is operably coupled to a plate 142 that can move up and down along a vertical axis of the cage and through the cage's opening 116 in the ceiling as the swivel mechanism 140 pivots along a hinge 146, as shown by the arrows 145 in FIG. 1A.

The cage is placed on the shelves so that the swivel is centered over it. In one embodiment, the swivel mechanism 140 is counterbalanced by weights 148, as shown in FIG. 1B, where the weights 148 placed at the other end of the plate opposite the cable 122. In one embodiment, the weights 148 are loosely screwed via non-concentric holes allowing easy balancing by just turning the weights 148 around which alters the torque they exert on the plate to the desired effect. The weights are adjusted so that the counterbalance arm holding them exerts a slightly higher torque than the side holding the swivel in a calibration step. In one embodiment, the torque exerted by the weights (60 grams, 10 cm from fulcrum) is about 0.058 Nm when the plate is in the horizontal position, and changes to about 0.029 Nm when the swivel moves all the way inside the cage and to about 0.048 Nm when the swivel reaches its highest position outside the cage (FIG. 1C). The torque exerted by the swivel (15 g, 15 cm from the fulcrum) and the EEG cable (10 g, 15 cm from the fulcrum) is 0.036 Nm when the plate is in the horizontal position. The torque exerted by the swivel and cable changes to 0.018 when the swivel is down inside the cage, and to 0.026 when the swivel is at its maximal upward position outside the cage, as shown in FIGS. 1B-1C). This system provides a minimal continuous upward tension that is equal in Newtons to the torque difference (Td) between the weights torque (Tw) and the swivel/cable torque (Tsc) divided by 0.15 m. In one embodiment, an additional correction factor of about 0.008 Nm is used to correct for the difference in arm's length of the balance system: Td=Tw−Tsc−0.008. In one embodiment, the upward tension T=Td/0.15A is on the headset when the animal is attached. The weights will generate a about 0.09 N upward tension force when the plate is in the horizontal position or when the swivel is at its highest upward position. In one embodiment, this upward tension force decreases to about 0.02 N when the swivel is pulled by the animal all the way down in the cage but is enough to prevent the swivel from remaining inside the cage when the animal ceases to pull down on it. The swivel therefore moves down when the animal pulls on it preventing downward tension-related disconnections, and it moves back up when the animal ceases to pull down which prevents cable folding inside the cage and thus cable exposure to rat's chewing. These modification allow long-term continuous EEG monitoring without premature disconnections of the EEG cable from the rat's head

As shown in FIG. 4, a rack system may include the cage and does not contain metal or other conductive material which provides the simplest and most efficient technique to electrically isolate rodents from each other and from environmental sources of electricity. This has markedly contributed to minimizing electrical artifact, which is a challenge commonly faced when simultaneously recording multiple animals. Nonconductive material was used including plexiglas and high-pressure laminate (HPL) to build the long-term rodent EEG monitoring unit, and paid a special attention to grounding and referencing in order to efficiently electrically shield rodents from ambient electrical noise, and to acquire high-quality artifact-free continuous tracings simultaneously on multiple rats. Nonconductive materials may include, but are not limited to: plastics, rubber, fiberglass, porcelain, ceramics, wood, glass, and the like.

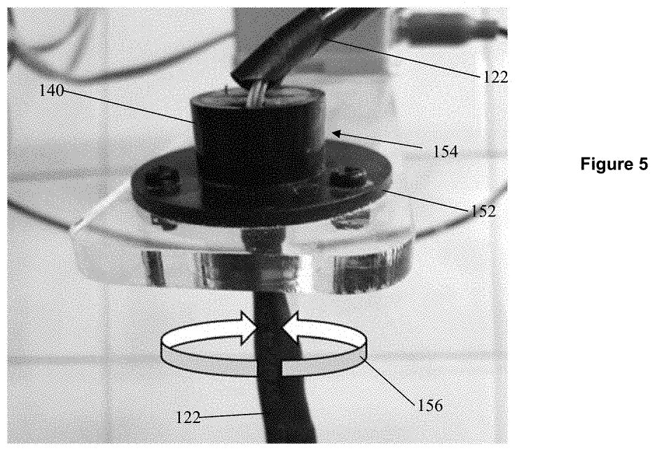

As shown in FIG. 5, the swivel mechanism 140 consists of a center rotating spindle 152 and six conductor rings 154 in electrical contact via conductor brushes. The swivel accommodates the EEG cable 122 carrying input from 6 channels in each rodent. In one embodiment, the swivel has a low torque (about 0.06 Nm) turning resistance preventing torsion of the EEG cable and subsequent stress on the rat's headset. This swivel 140 prevents cable 122 disconnection by allowing torsion-free motion of the rat in the horizontal plane. The white arrows 156 indicate the rotating mobile part.

The swivel mechanism is a connection that allows the cable to rotate horizontally. In one embodiment, the swivel mechanism may be a cylindrical rod that can turn freely within a support structure, where the rod is usually prevented from slipping out by a nut, washer or thickening of the rod. The cable can be attached to the ends of the rod or the center. Another embodiment of the swivel mechanism is a sphere that is able to rotate within a support structure, where the cable is attached to the sphere. Another embodiment of the swivel mechanism is a hollow cylindrical rod that has a rod that is slightly smaller than it's inside diameter inside of it and they are prevented from coming apart by flanges. The cable may be attached to either end.

The counterbalance mechanism operates as a lever consisting of a beam and fulcrum. In one embodiment, the plate 142 acts as the beam and a rectangular plate fixed to the rack's back to which the hinge is fixed acts as the fulcrum. The ideal lever does not dissipate or store energy, which means there is no friction in the hinge or bending in the plate. The power into the lever equals the power out, and the ratio of output to input force is given by the ratio of the distances from the fulcrum to the points of application of these forces. The mechanical advantage of a lever can be determined by considering the balance of moments or torque, T, about the fulcrum

T_{w}=F₁a, T₂=F₂ b; (1)

where F₁ is the input force to the lever and F₂ is the output force. The distances a and b are the perpendicular distances between the forces and the fulcrum.

Since the moments of torque must be balanced, T₁=T₂a. So, F₁a=F₂ b;

The mechanical advantage of the lever is the ratio of output force to input force,

MA=F₂/F₁−a/b. (2)

This relationship shows that the mechanical advantage can be computed from ratio of the distances from the fulcrum to where the input and output forces are applied to the lever, assuming no losses due to friction, flexibility or wear. This remains true even though the horizontal distance (perpendicular to the pull of gravity) of both a and b change (diminish) as the lever changes to any position away from the horizontal.

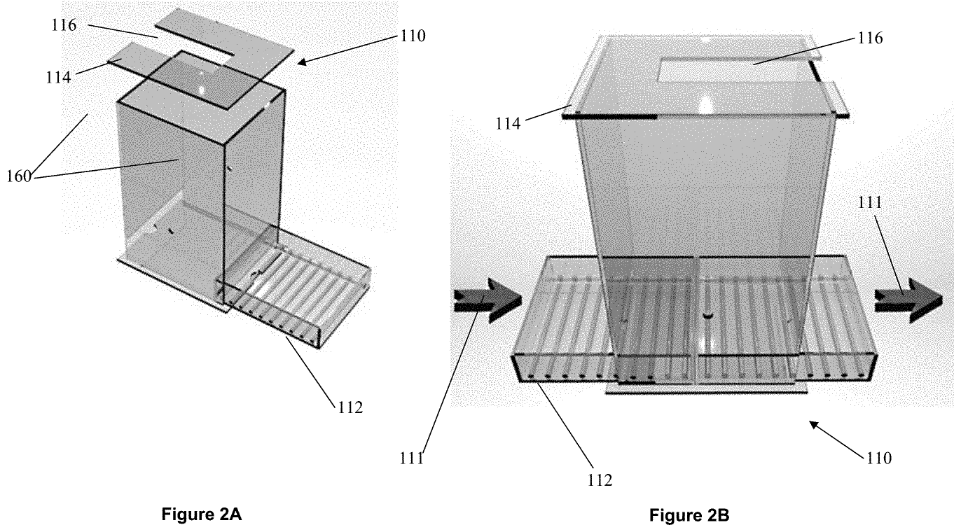

The EEG Swivel-Balance Cage System 100 comprises a rotating swivel mechanism 140 to prevent torsional tension on the EEG cable attached to the rat's head. The swivel mechanism is disposed on a vertically mobile plate that pivots along a hinge up and down through the cage ceiling. The swivel is counterbalanced with weights to provide a continuous gentle pull (upward tension ranges between about 0.02 to about 0.09 N depending on the angle of the pivoting plate) on the rodent's head preventing the EEG cable from folding inside the cage and thus exposure to rat's chewing. The cage 110 comprises a nonconductive Plexiglas material, as shown in FIGS. 2A-2B and a drawer floor 112 that traverse the bottom of the cage 110 and is moveable along an axis of the cage, shown by arrows 111, as to be completely removed from the cage 110. The drawer floor 112 allows the bedding and food changes without removing the rat from the cage, thus without interrupting prolonged continuous EEG recordings or other prolonged continuous animal recordings. This prevents the chronic wear and tear on the cable and electrodes, which introduces electrical noise (this is confirmed by the lack of discontinuity in the recorded EEG tracings) and ultimately loss of the electrical continuity required for adequate high quality EEG tracings. The cage 110 includes a top portion 114 with a horizontal cutout 116 to allow for the coupling of the plate 142 to freely move about the vertical axis with the swivel mechanism 140. In one embodiment, the drawer floor 112 is a gridded floor that contains the bedding and a side container for food. The gridded floor helps maintaining an even distribution of bedding over the floor. The food and bedding is replaced by sliding in a clean drawer 112 as the old one is pushed out. This system allows bedding and food changes without disconnecting the rat or removing it from the cage.

As described below, more than 100 rats were recorded in a rodent long-term monitoring unit without suffering from injuries or premature disconnections. The EEG Swivel-Balance Cage System provides safe and prolonged continuous EEG recordings in rodents, overcoming the notorious challenge of premature disconnections commonly faced with wired EEG techniques, and thus competing with the more advanced expensive wireless technologies.

As shown in FIGS. 1A-1B, the EEG Swivel-Balance Cage System 100 comprises a cage 110 and an EEG wiring system 120 including a cable 122 in contact with the rat's head through an interface 130. The cage 110 includes a swivel mechanism 140 which allows rotation (shown with the white arrows) of the EEG cable 122 as the rat moves in the horizontal plane. The swivel 140 can also move up and down through the cage's ceiling as it pivots along a hinge (double arrows) and is counterbalanced by weights (black arrow) always exerting on it a slight upward pull. The upward pull prevents twisting of the cable when the rat rises up. This simple yet key modification prevents both tension and torsion on the wires as the rat freely moves in both the horizontal and vertical planes. This has allowed long- term continuous EEG monitoring without running into premature disconnections and terminations of experiments, and thus prevented losses in budget, time and effort.

FIGS. 2A-2B. Rats are individually placed in customized plexiglas EEG recording cages (22L×22W×36H cm). These are equipped with sliding drawer-like gridded floors in which the bedding is easily changed by sliding a clean drawer in, while pushing the used one out. The drawer allows replacing the dirty bedding and providing food without removing the animal from the cage. Food is placed on the side of the gridded floor in a special container. The ceiling of the cage consists of a removable plexiglas lid which has a rectangular opening. This permits the vertical movement of a pivoting rectangular plate that holds a swivel above the cage and a weighted counterbalance on the other side as described above.

The special cage and swivel-balance system allows obtaining continuous long-term high signal to noise wired EEG for prolonged 2-4 months periods. It is simple to build and relatively markedly inexpensive compared to wireless technologies. 16 cages were built and obtained continuous 24-hour EEG data for months on 16 rats at a time (up to 100 recorded so far). This type of chronically recorded high quality data is essential for studying seizure chronobiology and mechanisms and for novel treatment discovery especially following brain insults that recapitulate epileptic disorders in children, a field that desperately need novel treatment strategies.

The cable may be a conduit that is a tube or duct for enclosing electric wires or optical cables or a channel or pipe for conveying water or other fluids. The conduit may be a channel, tube, or pipe for conveying fluids; in other contexts, conduit refers to a tube or duct for enclosing electrical connectors or wires, or for enclosing optical fibers. In certain embodiments, a multiplexed conduit comprises a plurality of channels, tubes, ducts, pipes, wires, and fibers suitably insulated from one another. In these instances, it is understood that the conduit communicates with multiple internal termini, as defined below.

The interface(s) operably coupled with the cable are used herein to mean one or multiple percutaneous, implanted, subcutaneous, intracerebral, intrapartioneal, intracisternal, intrathecal or intravenous conduits, catheters, wires or cannulas for use in infusion and/or withdrawal of fluids, and/or for electrical or optical input and output. The interface(s) could be a probe which is/are “indwelling,” which is an animal interface situated internal to the animal. Alternatively, the interface may comprise a “dwelling,” which is an animal interface dermally situated on the surface of the animal. The portion of the interface that is internal (or in contact with the animal in a dwelling system) to the animal is referred to herein as the indwelling segment, which terminates at an “internal terminus or multiple termini of an interface”, which is positioned in a vessel, organ, body cavity, or on the skin. The portion of the interface at which the conduit, catheter, wire or cannula passes through the skin (or is located distally from the skin in a dwelling system) of the animal, ends in the “external terminus of an interface”.

### EXAMPLES

Materials and Methods

II.1 Surgical Implantation of Epidural or Depth EEG electrodes

Surgeries and all animal care and postoperative treatments were approved by, and conducted in compliance with the guidelines of, the Institutional Animal Care and Use Committee (IACUC) at the American University of Beirut (AUB). The function of IACUC at AUB is in compliance with the public health service policy on the humane care and use of laboratory animals (USA), and adopts the guide for the care and use of laboratory animals of the Institute for Laboratory Animal Research of the National Academy of Sciences (USA).

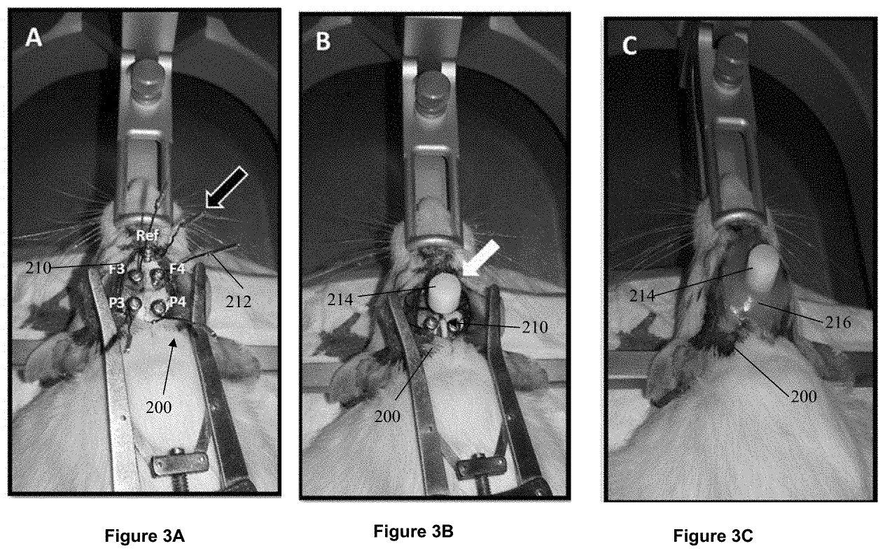

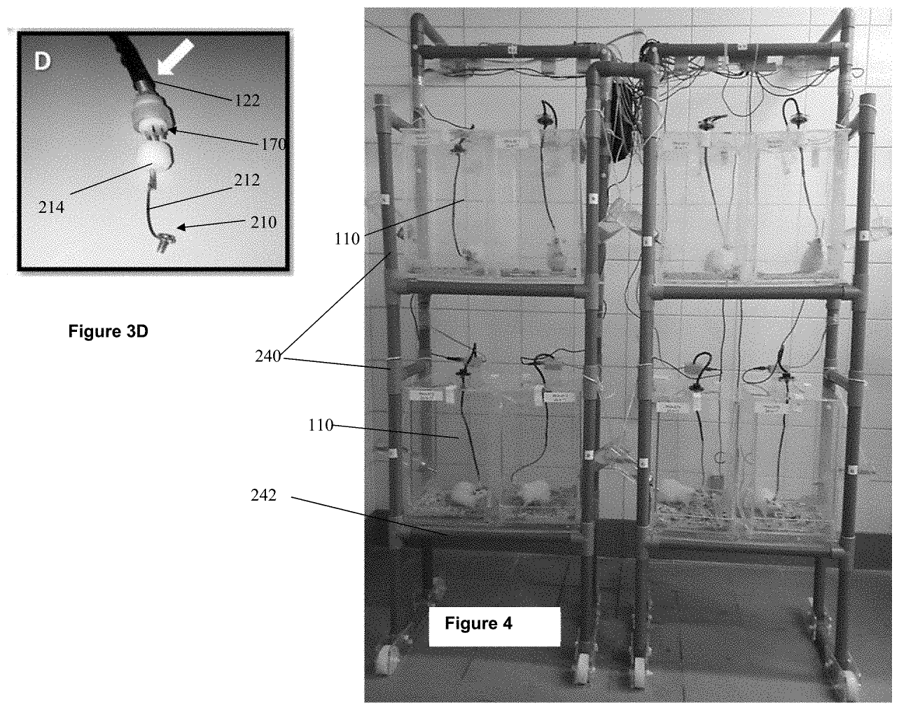

The following steps are followed in order to place epidural or depth electrodes, as shown in FIGS. 3A-3C.

1. The intramuscular surgical anesthesia mixture consists of ketamine (60 mg/kg), xylazine (6 mg/kg), and acepromazine (1.25 mg/kg).

2. After shaving the hair from the flat of the nose between the eyes down to the neck, the rat's head 200 is tightly secured on the stereotaxic frame (David Kopf Instruments, USA). Lubricating eye ointment is applied to prevent drying out and possible eye irritation from the used disinfectants.

3. The scalp is then sterilized with 10% iodine solution followed by 70% ethanol using cotton swabs through circular outward movements starting from the center of the shaved area. Using a sterile surgical blade, a 2 cm single midline incision is made starting from the bridge of the nose down to the posterior end of the cranium. The skin is held with a retractor, the calvarium is exposed, and bleeding controlled with cauterization and the application of pressure as needed. The exposed skull is then cleaned and dried using few drops of 3% hydrogen peroxide.

4. Once the skull surface is dry, 5 holes, one millimeter in diameter each, are made with a stereotaxic high-speed drill (David Kopf Instrument, USA) in order to place a combination of 5 epidural or depth electrodes 210 in the skull (including one reference electrode). The drill bit and the screw are matched in diameter size to ensure tight secure screw anchoring to the skull. Depending on the rat's age, epidural or depth electrodes are placed based on coordinates from the Sherwood and Timiras stereotaxic atlas of the developing rat brain or from the Paxinos and Watson adult rat brain atlas. The screws or depth electrodes are then attached to gold-plated stainless steel sockets (plastics one, USA) via insulated 1.5-2 cm copper wires 212. The 5 sockets are inserted in a 6 channel pedestal 214 (plastics one, USA), along with a 6^{th} socket attached to a free wire placed under the skin of the neck serving as a ground electrode. The pedestal and the screws are then covered with acrylic dental cement 216. Enough dental cement 216 is poured to fill the entire incision site giving the “headset” its final shape without requiring any skin suturing (FIG. 3A-3C).

5. The rats are placed on a warming pad (36-37° C.) during the entire surgery. After the surgery, they are placed in special customized single EEG cages for post-operative observation. Analgesia regimen with paracetamol is administered for 3 days postoperatively (1mg/m1 in drinking water).

6. After a 5 day recovery period, the EEG cables 122 ending with a 6 pin plug 170 (FIG. 3D) are attached to the 6 channel pedestal 214, and EEG recordings are initiated.

II.2 Customized EEG Cages and the Swivel-Balance System

II.2.1 Cages and Racks

Rats are individually placed in customized plexiglas EEG recording cages 110 (22L×22W×36H cm). These are equipped with sliding drawer-like gridded floors 110 in which the bedding is easily changed by sliding a clean drawer 112 in, while pushing the used one out (FIGS. 2A-2B). The drawer 112 allows replacing the dirty bedding and providing food without removing the animal from the cage (FIG. 2B). Food is placed on the side of the gridded floor in a special container. The ceiling of the cage consists of a removable plexiglas lid 114 which has a rectangular opening 116 (FIGS. 2A-2B). This permits the vertical movement of a pivoting rectangular plate 142 that holds a swivel mechanism 140 above the cage and a weighted counterbalance on the other side as described below. Similarly to the cages 110, the rack system 240 that houses them, are also built from electrically nonconductive material by assembling plastic pipes and connectors, providing electrical isolation for individual rats from each other and from sources of environmental electricity. Each rack 242 has 2 plexiglas shelves and is designed to accommodate at least 4 cages 110 (FIG. 4). Water bottles are held in place with plastic wires fastened to the rack, and are accessed by the rodents via holes drilled on the sidewall of the cages.

II.2.2 Swivel Balance and Cable System

A cage and wiring system is built with a special attention to the most fragile parts; the components connecting the headset to the swivel, especially those in contact with the rat's head. The swivel mechanism 140 allows rotation of the EEG cable 122 as the rat moves in the horizontal plane (FIG. 4). In addition to allowing the torsion-free animal motion in the horizontal plane provided by the swivel mechanism, the swivel-balance system also allows free movement in the vertical plane. Given that the rat's head movements are also vertical exerting pulling tension on the EEG cable, a swivel-balanced system that can move up and down to prevent tension when the rat is in the resting position, and also preventing cable twisting inside the cage when the rat moves up. This was achieved by fixing the swivel on a rectangular plate 142 that pivots vertically along a hinge neck placed on the back wall of the rack allowing the swivel to move up and down through the rectangular opening in the ceiling (FIG. 1A). The plate pivots along the hinge placed in its middle with one arm holding the swivel above the center of the cage on one side, and an external arm holding counterbalancing weights on the other side. These weights are placed on the external arm of the plate so that the weights of the cable and swivel of the inner arm are counterbalanced. This allows the plate holding the swivel to return to its neutral position (in the horizontal plane of the cage ceiling) when the rat is resting (FIG. 1B). In addition, the EEG cables connecting the headset to the swivel inside the cage are also protected against chewing with stainless steel spring covers. The length of the EEG cable hanging inside the cage is 32 cm (swivel to rat's headset). This length was calculated based on the cage dimensions in order to prevent cable tension when the rat is in the corner, and at the same time to avoid excessive cable folding and thus increased exposure to chewing when the rat is in the center of the cage.

II.3 Digital EEG Recording System

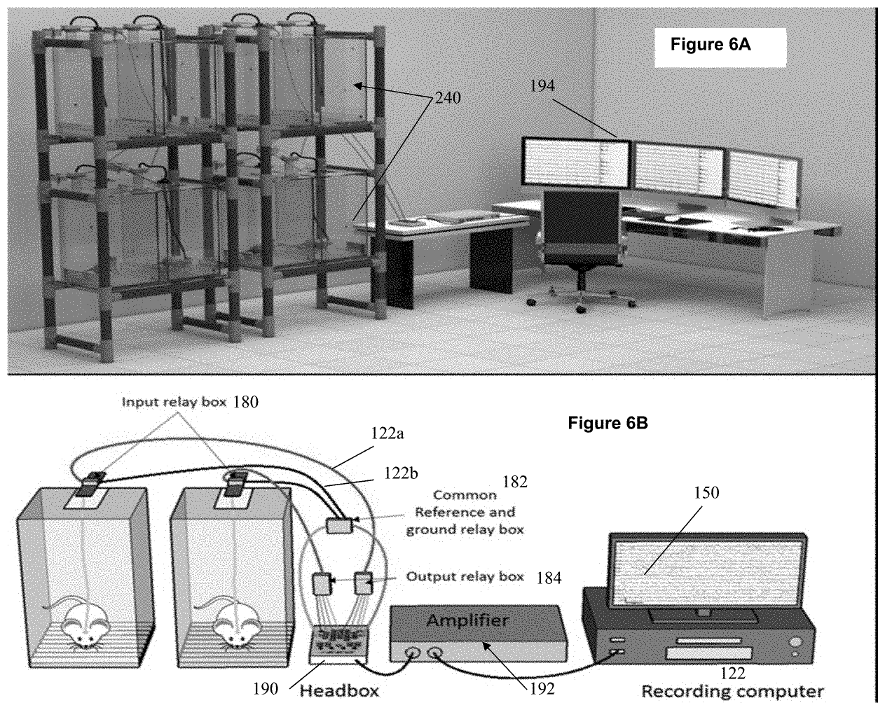

In order to simplify the setup, the swivel's input relay box 180 is connected to a common reference and ground relay box 182 through wires 122b, and to an output relay box 184 through wires 122b. Both the common reference and the ground relay box 182 and the output relay box 184 are connected to the EEG recording headbox 190 (32 channel headbox, XLTEK, USA) as described in details in FIGS. 6A-6B. Relay boxes 180 are used in order to minimize the number of wires which not only improves organization but also the ability to readily access cables 122 and to systematically troubleshoot the various components of the system when needed. The four epidural cortical electrodes or depth electrodes originating from one rat's headset are ultimately connected to four different ports on the 32 channel headbox 190 which can accommodate up to 8rats. The reference electrodes of the 8 animals are daisy chained and connected to the only available reference port in the headbox 190. The 8 ground electrodes are also approached in the same manner and connected to the single ground port on the headbox. The headbox feeds data into an amplifier 192 that is connected to the recording computer 122. This setup allows the simultaneous continuous long-term recording of 8 animals using one headbox. The grounding and referencing approaches are designed to maximize the signal to noise ratio while overcoming the limitations of the medical headbox that allows input from only one reference electrode and only one ground electrode as it is designed for a single human subject. In addition to the bipolar transverse and bipolar longitudinal montages, the laplacian montage option is used to create an average referential montage for each rat (comparing the input of each of the four epidural or depth electrodes from one animal to the average of these four). Chronic continuous EEG tracings are briefly discontinued and restarted on the recording computer every 48-72 hours in order to minimize EEG file sizes and facilitate the organization of the sizeable EEG data review process.

As shown in FIG. 6B, the output relay box 184 receives wiring from cables 122a, which carry the epidural cortical or depth electrode signal (˜4 per rat), while the common reference and ground relay box 182 receives wiring from cables 122b carrying signal from the ground and reference electrodes. The output relay box 184 splits again the electrical signal of each rat into its 4 epidural cortical or depth electrode wires, and these are connected to 4 ports on the headbox 190. Given that a single reference port and a single ground port are available on the digital 32 channel headbox 190 (Xltek, Natus Medical, USA), the common reference/ground relay box 182 combines reference and ground electrode signals from 8 rats into one common reference and one common ground respectively, which are placed in their respective headbox ports. The headbox 190 feeds data into an amplifier 192 that is connected to the recording computer 122. The used cable system and relay boxes minimize the number of wires for easy access and troubleshooting if needed .EEG tracings can be acquired simultaneously on 8 rats using one computer recorder and one headbox. Only two cages are shown for illustrative simplicity.

### III. ILLUSTRATIVE RESULTS AND DISCUSSION

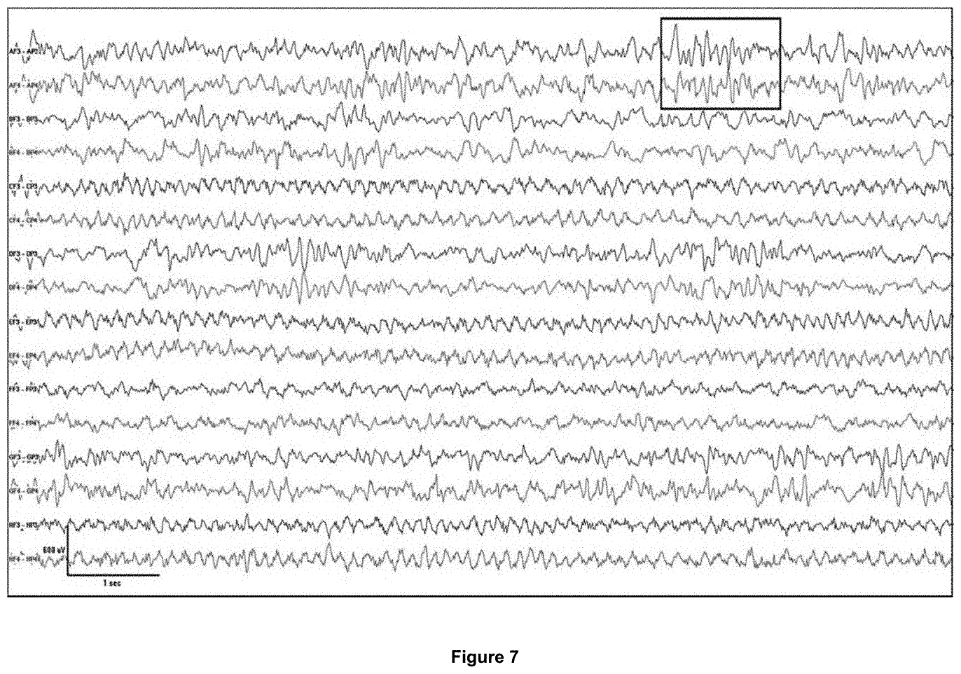

Using the herein described long-term EEG monitoring unit setup, chronic EEG simultaneously was performed on up to 16 rats (about 8 rats per EEG recorder) without suffering from interruptions or EEG cable disconnections for several months. The recording can be performed with or without continuous video. More than 100 rats have been recorded and only experienced two detachments due to cable chewing by large rats, late in the EEG recording, at 140 days of age. The whole EEG cable hanging inside the cage may be covered with a protective metal spring cover (as opposed to only the lower third of the cable in the initial setup). In one study, a three months of recording was conducted on all 60 experimental rats. The recordings are of high quality with minimal artifact from movement or ambient electricity (FIGS. 7-9). No injuries to rats were encountered.

FIG. 7 is a screenshot of a continuous EEG recording performed simultaneously on 8 rats using one EEG recorder. The 8 rats display allows the time-efficient review of sizeable data spanning months of EEG recordings. Each alphabetical letter refers to a rat subject. Shown is a longitudinal bipolar montage with 2 channels per rat (F3: left frontal, P3: left parietal, F4: right frontal, P4: right parietal). This EEG reveals a normal tracing for all the rats. Tracing of rats C, E, and H are consistent with the 6 Hz theta rhythms seen in relaxed rodents, likely a thalamocortical feature that recapitulates the human posterior dominant rhythm given the rhythmicity and monomorphism. EEG of rats A, B, D, F and G shows drowsy and sleep rhythms with intermittent spindle activity in rat A (black rectangle). The notch was set at 50 Hz.

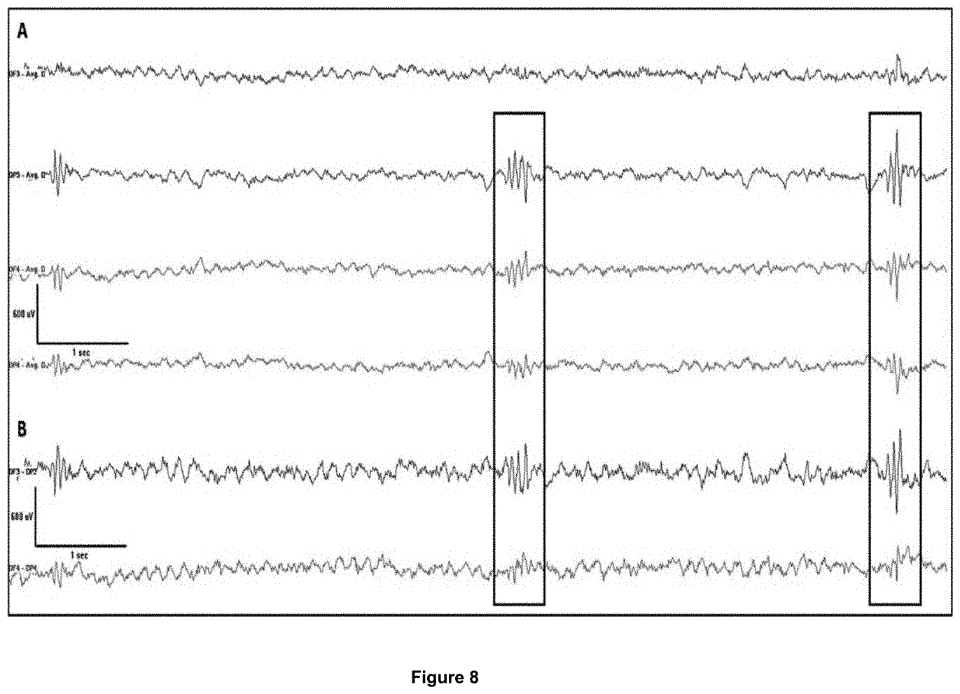

FIG. 8 is an illustrative image of a tracing in an average referential montage (panel A) and in a longitudinal bipolar montage (panel B). Left hemispheric spikes are seen with a maximum in the left parietal region (black rectangle). F3: left frontal, F4: right frontal, P3: left parietal, P4: right parietal, Avg: average of the 4 electrodes.

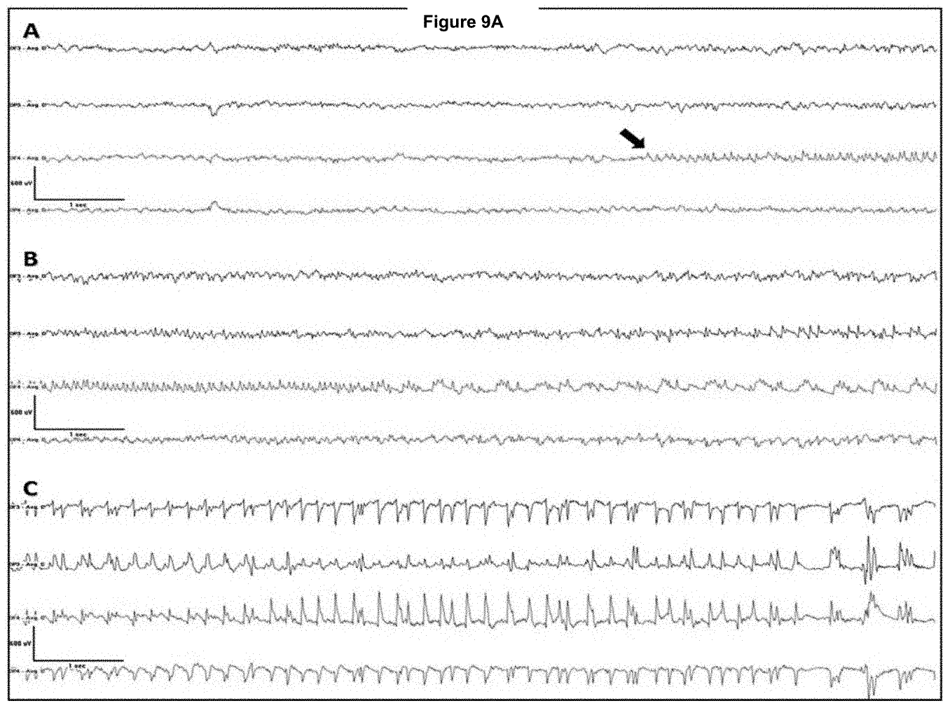

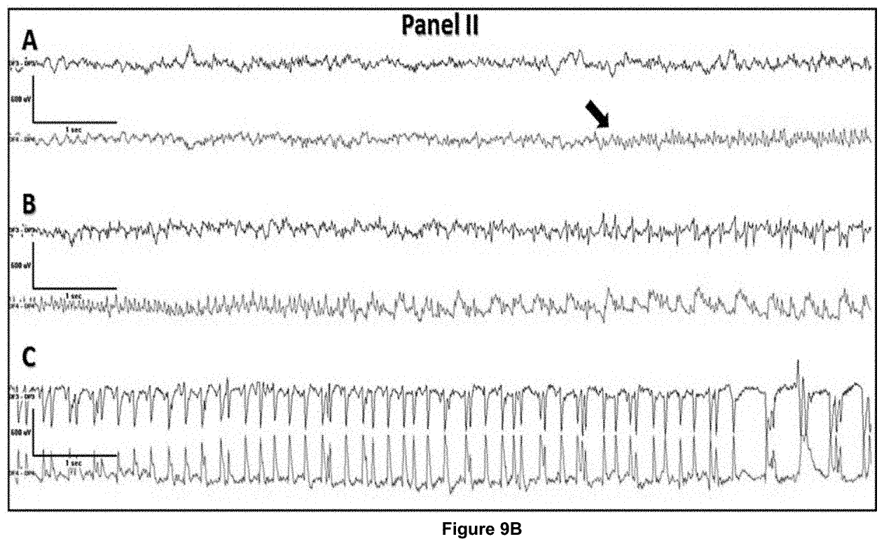

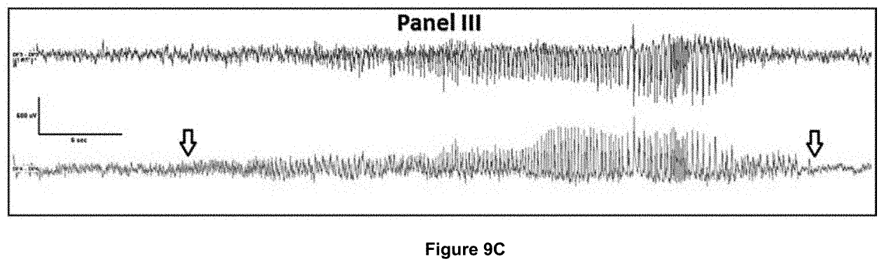

FIG. 9A are illustrative successive screenshots (9A to C) of a right frontal seizure in an average referential montage (FIG. 9A) and in a longitudinal bipolar montage (FIG. 9B). A typical seizure with “a rhythmicity that evolves in space and time” is shown with clarity in this rat. The seizure starts in the right frontal area (black arrow) as shown in screenshot A in both FIGS. 9A-9B, then progresses to involve the right hemisphere with slower rhythmic activity (screenshot B in both FIGS. 9A-9B), and finally clearly involves the left hemisphere with bilateral rhythmic spikes (screenshot C in both FIGS. 9A-9B). The whole seizure is shown in FIG. 9C (white arrows indicate onset and offset). F3: left frontal, F4: right frontal, P3: left parietal, P4: right parietal, Avg: average of the 4 electrodes.

Herein, the method and rodent EEG monitoring unit setup records continuous EEG simultaneously on multiple animals for several months without putting the rats at risk of injuries and without EEG cable disconnections and premature experimental terminations. The improvements applied to the wired cage system and the attention to details allowed long-term EEG recording performance without using wireless radiotelemetry; a technology that remains very expensive at this point in time especially in countries outside the United States and Europe. Special attention to the most vulnerable parts of the EEG recording system, namely the components between the rat's head and the swivel was key. Indeed, the vertically mobile swivel-balance system prevents torsion due to motion in the horizontal plane as well as tension imparted by vertical movements of rats. The delicate balancing of the counterweights was also key in providing a continuous gentle upward pull on the rat's headset, preventing the folding of the EEG cable inside the cage which exposes it to the natural tendency of rodents to chew on it. In addition, tightly securing the screw electrodes to the calvarium was essential, since the screws serve as the main anchors that attach the EEG cable to the rat's head. On the other hand, the dental cement is merely the “glue” that keeps together the electrodes, wires, and other components of the headset and shield them from the external environment. The use of a sliding drawer approach to change the bedding and place food while the rat remained connected to the EEG cable inside the cage, minimized frequent EEG cable connections and disconnections, and attenuated the mechanical wear and tear of the cables and headset. This has also contributed to maintaining the integrity of electrical continuity and in improving the quality and longevity of our continuous EEG recordings.

In addition to markedly attenuating the various sources of mechanical disruptions to electrical continuity, the attention to electrode wiring and grounding also aided in obtaining the high quality artifact-free EEG tracings. The use of nonconductive material in almost all the elements of the long-term EEG rodent unit, including the plexiglas cages, plastic rack system, HPL trays and HPL computer recorder tables, has facilitated the grounding process and the electrical shielding of the rats from ambient sources of electrical noise. Grounding and referencing via a daisy chain approach in this electrically shielded unit was performed without difficulties, overcoming the commonly challenging task of simultaneously recording multiple animals using medical devices tailored to one human subject.

### IV. CONCLUSIONS

The herein described rodent long-term EEG monitoring unit provides a simple and cost-effective approach to acquire continuous high-quality EEG tracings simultaneously on multiple rats for several months without disconnections or injuries. The customized cages, housing environment, and the swivel-balance system can be easily constructed using relatively inexpensive materials and tools. The described methodological approach can also be easily implemented with special attention to the details of electrode implantation and wiring. Our simple and inexpensive, yet key modifications result in a markedly improved traditional hardwired EEG and provide the necessary quality and amount of data required in translational epilepsy research at a much lower cost than more advanced wireless systems. While this applies to translational laboratory in general, it is particularly true for many laboratories operating outside the United States and Europe, as they usually suffer from limited access to advanced technologies.

### REFERENCES

(1) Noachtar S, Remi J. The role of EEG in epilepsy: a critical review. Epilepsy & Behavior 2009; 15(1):22-33.

(2) Appavu B, Fox J, Little R, Kerrigan J F, Obeid M. Seizures Induced by Exiting Water: A Unique Form of Reflex Epilepsy. Neurologist 2017 September; 22(5):196-198.

(3) Assi L, Saklawi Y, Karam P E, Obeid M. Treatable Genetic Metabolic Epilepsies. Curr Treat Options Neurol 2017 September; 19(9):30-017-0467-0.

(4) Leiser S C, Dunlop J, Bowlby M R, Devilbiss D M. Aligning strategies for using EEG as a surrogate biomarker: a review of preclinical and clinical research. Biochem Pharmacol 2011; 81(12):1408-1421.

(5) Bertram E H, Williamson J M, Cornett J F, Spradlin S, Chen Z F. Design and construction of a long-term continuous video-EEG monitoring unit for simultaneous recording of multiple small animals. Brain Research Protocols 1997; 2(1):85-97.

(6) Mountney A, Shear D A, Potter B, Marcsisin S R, Sousa J, Melendez V, et al. Ethosuximide and phenytoin dose-dependently attenuate acute nonconvulsive seizures after traumatic brain injury in rats. J Neurotrauma 2013; 30(23):1973-1982.

(7) Dudek F E, Staley K J. The time course of acquired epilepsy: implications for therapeutic intervention to suppress epileptogenesis. Neurosci Lett 2011; 497(3):240-246.

(8) Bolkvadze T, Pitkanen A. Development of post-traumatic epilepsy after controlled cortical impact and lateral fluid-percussion-induced brain injury in the mouse. J Neurotrauma 2012; 29(5):789-812.

(9) Lu X M, Shear D A, Graham P B, Bridson G W, Uttamsingh V, Chen Z, et al. Dual therapeutic effects of C-10068, a dextromethorphan derivative, against post-traumatic nonconvulsive seizures and neuroinflammation in a rat model of penetrating ballistic-like brain injury. J Neurotrauma 2015; 32(20):1621-1632.

(10) Obeid M, Frank J, Medina M, Finckbone V, Bliss R, Bista B, et al. Neuroprotective effects of leptin following kainic acid-induced status epilepticus. Epilepsy & Behavior 2010; 19(3):278-283.

(11) Pitkanen A, Lukasiuk K, Dudek F E, Staley K J. Epileptogenesis. Cold Spring Harb Perspect Med 2015 Sep. 18; 5(10):10.1101/cshperspect.a022822.

(12) van Luijtelaar G, Luttjohann A, Makarov V V, Maksimenko V A, Koronovskii A A, Hramov A E. Methods of automated absence seizure detection, interference by stimulation, and possibilities for prediction in genetic absence models. J Neurosci Methods 2016 Feb. 15; 260:144-158.

(13) Sarkisian M R. Overview of the current animal models for human seizure and epileptic disorders. Epilepsy & Behavior 2001; 2(3):201-216.

(14) Loscher W. Animal models of epilepsy for the development of antiepileptogenic and disease-modifying drugs. A comparison of the pharmacology of kindling and post-status epilepticus models of temporal lobe epilepsy. Epilepsy Res 2002; 50(1):105-123.

(15) Williams P A, Dou P, Dudek F E. Epilepsy and synaptic reorganization in a perinatal rat model of hypoxia-ischemia. Epilepsia 2004; 45(10):1210-1218.

(16) Morimoto K, Fahnestock M, Racine R J. Kindling and status epilepticus models of epilepsy: rewiring the brain. Prog Neurobiol 2004; 73(1):1-60.

(17) Williams P, White A, Ferraro D, Clark S, Staley K, Dudek F E. The use of radiotelemetry to evaluate electrographic seizures in rats with kainate-induced epilepsy. J Neurosci Methods 2006; 155(1):39-48.

All publications and patent applications mentioned in this specification are herein incorporated by reference to the same extent as if each individual publication or patent application was specifically and individually indicated to be incorporated by reference.

System

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Software includes applications and algorithms. Software may be implemented in a smart phone, tablet, or personal computer, in the cloud, on a wearable device, or other computing or processing device. Software may include logs, journals, tables, games, recordings, communications, SMS messages, Web sites, charts, interactive tools, social networks, VOIP (Voice Over Internet Protocol), e-mails, and videos.

In some embodiments, some or all of the functions or process(es) described herein and performed by a computer program that is formed from computer readable program code and that is embodied in a computer readable medium. The phrase “computer readable program code” includes any type of computer code, including source code, object code, executable code, firmware, software, etc. The phrase “computer readable medium” includes any type of medium capable of being accessed by a computer, such as read only memory (ROM), random access memory (RAM), a hard disk drive, a compact disc (CD), a digital video disc (DVD), or any other type of memory.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A cage with a swivel-balance system for long-term monitoring of an animal comprising:
a. a swivel on a vertically mobile plate that pivots along a hinge up and down through a cage ceiling; wherein the swivel is counterbalanced with weights to provide a continuous gentle pull on an interface coupled with the animal as to prevent a cable from folding inside the cage.

2. The cage with a swivel-balance system of claim 1, wherein the cage is made with a nonconductive material and the cage includes a drawer floor to allow bedding and food changes without removing the animal from the cage and without interrupting prolonged continuous animal recordings.

3. An counterbalance system to accommodate an animal movements in a vertical plane:
comprising:
a. Preventing tension on a cable coupled to an animal's head when the animal moves down in a cage;
b. Preventing the cable from folding inside the cage when the animal moves up;
c. Pivoting the cable from a plate on a first end with a swivel centered above the cage; and
d. Counterbalancing the plate on a second end with weights slightly heavier than the cable to provide a continuous gentle upward pull on the animal's head.

4. The counterbalance system of claim 3, further comprising providing the cage with a drawer floor to allow bedding and food changes without removing the animal from the cage and without interrupting prolonged continuous animal recordings.
